(19) United States
(12) Patent Application Publication
(10) Pub. No.: US 2007/0224660 A1
(43) Pub. Date: Sep. 27, 2007

(54) Method for Assessment of Cytotoxic T Lymphocyte Activity

(76) Inventors: Yan Cui, New Orleans, LA (US); Kong Chen, New Orleans, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/578,477

(22) PCT Filed: Apr. 14, 2005
(86) PCT No.: PCT/US05/13120

§ 371 Date: Feb. 28, 2007

Related U.S. Application Data
(60) Provisional application No. 60/563,043, filed on Apr. 16, 2004.

(51) Int. Cl. C12Q 1/06

(52) U.S. Cl. 435/39

(57) ABSTRACT

A new cytotoxic T lymphocyte (CTL) assay has been discovered using two cell lines that stably express either green fluorescent protein (GFP) or red fluorescent protein (DsRed), which are distinguishable by FACS, fluorescence microplate reader, or fluorescence microscopy. Using one cell line as a target (T) to present antigen and the other, at the same number, as an internal control (reference, R), a new CTL assay (named fluorolysometric (FL)-CTL assay) way developed based on cytolysis of these fluorescent protein-expressing targets detectable by FACS. This FL-CTL assay was further extended for use with a fluorescent microplate reader. This FL-CTL assay was reproducibly used to determine primary CTL activity at high sensitivity when compared to other conventional assays with in vivo activated T cells against different antigens. This new reliable, sensitive, convenient, and economical CTL assay has broad application potentials for experimental and clinical use in different antigen and effector-target systems.

The benefit of the filing date of provisional U.S. application Ser. No. 60/563,043, filed 16 Apr. 2004, is claimed under 35 U.S.C. § 119(e) in the United States, and is claimed under applicable treaties and conventions in all countries.

### TECHNICAL FIELD

This invention pertains to a method to measure cytotoxic T lymphocyte (“CTL”) activity based on cell-mediated cytolysis of fluorescent protein (e.g., green or red) expressing cells quantified by a common fluoro-based method, e.g., flow cytometry or fluorescence microplate-reader.

### BACKGROUND ART

Cytotoxic T lymphocytes (“CTL”) are important effectors in host immune responses to tumors, intracellular pathogens and transplant rejection. This cytotoxicity is based on cell-surface antigen recognition and mediated through either release of perforin and granzyme containing cytolytic granules or engagement of cell surface death receptors. See M. Barry et al., “Cytotoxic T lymphocytes: all roads lead to death,” Nat. Rev. Immunol., vol. 2, pp. 401-409 (2002); and J. Lieberman, “The ABCs of granule-mediated cytotoxicity: new weapons in the arsenal,” Nat. Rev. Immunol., vol. 3, pp. 361-370 (2003). An efficient and accurate evaluation of CTL function with a highly sensitive and convenient assay is important not only in clinical assessment of immune dysfunction, but also for development and evaluation of therapeutic efficacy of cancer immunotherapy, viral infection and immune suppressive regimens to minimize transplant rejection.

Currently, several assay systems are widely used to evaluate CTL activity based on the direct measurement of target cell killing or indirect parameters. The chromium (⁵¹Cr) release assay, which directly measures cytolytic activity as ⁵¹Cr radioactivity released from killed target cells, is the most widely used CTL assay since its development. See K. T. Brunner et al., “Quantitative assay of the lytic action of immune lymphoid cells on 51-Cr-labelled allogeneic target cells in vitro; inhibition by isoantibody and by drugs,” Immunology, vol. 14, pp. 181-96 (1968). Likewise, the JAM test directly evaluates CTL killing by measuring reduction in radioactivity of ³H-thymidine pre-incorporated in genomic DNA from the remaining target cells not eliminated by cytotoxic T cells. See P. Matzinger, “The JAM test. A simple assay for DNA fragmentation and cell death,” J. Immunol. Methods, vol. 145, pp. 185-92 (1991). Both assays measure the total radioactivity released from target cells, and both may have limited sensitivity for determining CTL activity of in vivo activated cells. Under physiological conditions, antigen-specific CTL-effector frequency of most immune responses is so low that the CTL activity often can not be reliably measured using these conventional methods without further in vitro stimulation. Therefore, considerable efforts have been made to improve the sensitivity of CTL assays and to determine CTL function at the single-cell level. See L. L. Carter et al., “Single cell analyses of cytokine production,” Curr. Opin. Immunol., vol. 9, pp. 177-182 (1997); and C. Ewen et al., “A novel cytotoxicity assay to evaluate antigen-specific CTL responses using a calorimetric substrate for Granzyme B,” J. Immunol. Methods, vol. 276, pp. 89-101 (2003). Towards this end, the ELISPOT (enzyme-linked immunospot) assay and intracellular cytokine staining have been developed to enumerate interferon-γ (or other cytokine) producing cells as estimates of CTL effector frequency. See Carter et al., 1997; and Y. Miyahira et al., “Quantification of antigen specific CD8+ T cells using an ELISPOT assay,” J. Immunol. Methods, vol. 181, pp. 45-54 (1995). However, these assays often involve lengthy procedures. But more importantly, these cytokine-producing cells may not truthfully represent cells with immediate cytolytic function (CTL effector). See J. E. Snyder et al., “Measuring the frequency of mouse and human cytotoxic T cells by the Lysispot assay: independent regulation of cytokine secretion and short-terin killing,” Nat Med., vol. 9, pp. 231-235 (2003). Likewise, development in tetramer technology significantly improved the capability of detecting and enumerating antigen-specific cytotoxic T lymphocytes, but did not provide a function determination. See J. D. Altman et al, “Phenotypic analysis of antigen-specific T lymphocytes,” Science, vol. 274, pp.94-96 (1996).

Lately, flow-cytometry (FACS) based systems have been explored to directly evaluate antigen-specific cytolysis using target cells labeled with fluorescent dye or protein, or incorporated with fluorogenic caspase substrates. See N. G. Papadopoulos et al., “An improved fluorescence assay for the determination of lymphocyte-mediated cytotcoxicity using flow cytometry,” J. Immunol. Methods, vol. 177, pp. 101-111 (1994); H. Lecoeur et al., “A novel flow cytometric assay for quantitation and multiparametric characterization of cell-mediated cytotoxicity,” J. Iminunol. Methods, vol. 253, pp. 177-87 (2001); M. Hoppner et al., “A flow-cytometry based cytotoxicity assay using stained effector cells in combination with native target cells,” J. Immunol. Methods, vol. 267, pp. 157-163 (2002); N. Kienzle et al, “The fluorolysis assay, a highly sensitive method for measuring the cytolytic activity of T cells at very low numbers,” J. Imrnunol. Methods, vol. 267, pp. 99-108 (2002); M. R. Betts et al., “Sensitive and viable identification of antigen-specific CD8+ T cells by a flow cytometric assay for degranulation,” J. Immunol. Methods, vol. 281, pp. 65-78 (2003); A. Chahroudi et al., “Measuring T cell-mediated cytotoxicity using fluorogenic caspase substrates,” Methods, vol. 31, pp. 120-126 (2003); T.S. Hawley et al, “Four-color flow cytometric detection of retrovirally expressed red, yellow, green and cyan fluorescent proteins,” CLONTECHniques, July, 2001 (www.clontech.com); L. Cheng et al., “Analysis of GFP and RSGFP expression in mammalian cells by flow cytometry,” CLONTECHniques, October, 1995 (www.clontech.com); I. F. Hermans et al, “The VITAL assay: a versatile fluorometric technique for assessing CTL- and NKT-mediated cytotoxicity against multiple targets in vitro and in vivo,” J. Immunol. Methods, vol. 285, pp. 25-40 (2004); U.S. Patent Application 2002/0115157, and U.S. Pat. No. 6,828,091. The CTL activity was determined as a decrease in viable fluorophore-labeled cells or an increase in caspase substrate-positive cells. While these assays have been proven to be efficient in detecting cytolytic killing at the single-cell level, a major limitation on accurate determination of CTL activity is that they heavily rely on accurate enumeration of total apoptotic target cells of various stages and forms, which can be complicated especially with the extended duration of CTL assays. Since FACS is more appropriate in determining relative percentages of specific populations instead of actual numbers of cells, total acquisition and accurate enumeration of absolute numbers of viable or apoptotic cells by FACS, in the absence of an internal control (reference), is very cumbersome. In fact, this issue was addressed in some of the recent modifications with the introduction of the same number of distinct fluorescent dye-labeled particles, or with prime-boost immunization. See M. J. Estcourt et al., “Prime-boost immunization generates a high frequency, high-avidity CD8(+) cytotoxic T lymphocyte population,” Int. Immunol., vol. 14, pp. 31-37 (2002); and Kieizle et al., 2002.

### DISCLOSURE OF INVENTION

We have developed a new cytotoxic T lymphocyte (CTL) assay by taking advantage of efficient and long-term transgene delivery of lentiviral vectors to a wide variety of cells and establishing two cell lines that stably express either green fluorescent protein (GFP) or, red fluorescent protein (DsRed), which are distinguishable by FACS, fluorescence microplate reader, or fluorescence microscopy. Using one cell line as a target (T) to present antigen and the other, at the same number, as an internal control (reference, R), we have developed a new CTL assay based on cytolysis of these fluorescent protein-expressing targets detectable by FACS. The new assay is named a fluorolysometric (FL)-CTL assay. Of particular significance, this FL-CTL assay can also be carried out with a more efficient and convenient fluorescence microplate reader-based determination using only target cells and CTL cells achieving sensitivity comparable to the FACS-based method. This FL-CTL assay was reproducibly used to determine primary CTL activity at high sensitivity when compared to other conventional assays with in vivo activated T cells against different antigens. In addition, this new reliable, sensitive, convenient, and economical CTL assay has broad application potentials for experimental and clinical use in different antigen and effector-target systems.

### DESCRIPTION OF THE DRAWINGS

FIG. 1A illustrates a schematic illustration of the two lentiviral vectors used for transducing P815 cells: on the left is an illustration of cppt.EF.GFP (the green fluorescence protein) and on the right is an illustration of cppt.EF.DsRed (red fluorescence protein).

FIG. 1B illustrates P815 cells after more than one year after transduction with either cppt.EF.GFP (left) or cppt.EF.DsRed (right) before (top) and after sorting (bottom) to a purity of more than 98% of GFP⁺ or DsRed⁺ expressing cells by FACS.

FIG. 2A illustrates the effects of GFP or DsRed expression on viability and metabolic activity of the lentiviral vector transduced P815 cells compared with wild type P815 (control), as measured by optical density (O.D.) of a metabolized MTT substrate.

FIG. 2B illustrates the effects of GFP or DsRed expression on long-term growth of the lentiviral vector transduced P815 cells compared with wild type P815 (control), as measured by growth in numbers plotted against time in culture for a total of 3 weeks with a 1:5 subculture every 3 or 4 days.

FIG. 3 illustrates FACS plots of stable P815 cell lines expressing GFP or DsRed loaded with HA specific MHC I peptide and used as targets (T) (GFP-HA, top panel, and DsRed-HA, bottom panel) mixed with reference cells (R) (P815-DsRed (top panels) or P815-GFP (bottom panels) at a T:R ratio of 1:1, and incubated for 4 hours with activated HA specific CTLs (as effector cells (E)) at a E:T ratio of 0:1 (left panels), 1:1 (middle panels), and 10:1 (right panels).

FIG. 4A illustrates FACS plots that represent the activation status (expressed as up-regulation of CD44 and down-regulation of CD62L expression as compared to non activated cells (NT naïve)) of T cells from non-transgenic mice (NT) activated by three-day culture with Con A stimulation (NT-Con A activated) and HA specific CTL effector cells activated by three-day culture with MHC-I HA peptide (Clone4-HA-activated).

FIG. 4B illustrates a comparison of HA specific GFP⁺ P815 target cell killing at various E:T ratios using the conventional JAM test and the FL-CTL assay, using the activated HA-specific cells or non-specific T cells of FIG. 4A.

FIG. 4C illustrates a comparison of HA specific GFP+ P815 target cell killing at two E:T ratios (0:1 and 10:1) using the conventional ⁵¹Cr-release assay and the FL-CTL assay, using the activated HA-specific cells or non-specific T cells of FIG. 4A.

FIG. 4D illustrates a comparison of HA specific GFP+ P815 target cell killing at various E:T ratios and using the activated HA-specific cells or non-specific T cells of FIG. 4A, as assayed with the FL-CTL assay after an incubation time of 4 hr and 24 hr.

FIG. 5A illustrates the relationship between cell number and fluorescent intensity of GFP⁺ P815 cells based on fluorescence reading as determined with a microplate fluorescence reader.

FIG. 5B illustrates the relationship between cell number and fluorescent intensity of P815 cells with DsRed⁺ as read using a microplate fluorescence reader.

FIG. 5C illustrates a comparison between the CTL activity using a fluorescent microplate reader-based detection and the FACS-based system, using DsRed⁺ P815 cells as targets in both assays.

FIG. 6A illustrates a comparison between the hemagglutinin (HA)-specific CTL activity determined by the FL-CTL assay and a ⁵¹Cr release assay of CTL cells collected from non-immunized naïve mice and Vacc-HA immunized (HA-primed) mice that also received HA-specific CD8 cells from transgenic mice.

FIG. 6B illustrates the hemagglutinin (HA)-specific CTL activity of endogenous T cells activated by Vacc-HA in cells from immunized (Primed) and non-immunized (Naïve) BALB/c mice.

FIG. 6C illustrates GFP specific CTL activity in splenocytes harvested from BALB/c mice immunized with GFP-expressing bone marrow derived dentritic cells (GFP-BMDC), using the FL-CTL assay with GFP⁺ P815 cells as target cells and DsRed⁺ P815 cells as reference.

### MODES FOR CARRYING OUT THE INVENTION

Most of the currently used CTL assays, including recently improved FACS-based assays, heavily rely on pre-loading or labeling target cells with radioactive chemicals, such as ⁵¹Cr and ³H, or fluorogenic reagents. Besides environmental safety concerns and inconvenience, variation of each labeling condition further hinders their reproducibility and sensitivity. This invention took advantage of the efficient and stable gene transfer using lentiviral vectors and established cell lines, each expressing a different fluorescent-colored protein, e.g., green fluorescent protein (GFP) or red fluorescent protein (DsRed). The technology as described herein provides a basis for improving the reliability, accuracy, and convenience of FACS-based CTL analysis by the introduction of at least two or more similar, yet fluorophoric distinct cell lines, with one cell line as an internal reference (R) and the remaining cell lines as targets (T). Therefore, antigen-specific cytolysis is determined based on a relative ratio of T to R cells, not on the absolute number of total remaining target cells. Additionally, the antigen loading to target cells can be Teplaced with lentiviral-mediated permanent antigen gene transfer to target cells. The use of target cells transduced with an entire antigen gene or mini-gene (e.g., selected epitope of a specific antigen) provides flexibility in examining “bulky” CTL function against multi-epitopes of the same antigen or an individual epitope, respectively, depending on needs. Therefore, this new FL-CTL assay system represents a convenient, sensitive, reliable and economical approach to assess CTL activity. Using different fluorescent proteins that can spectrally be distinguished, multiple antigen target cell lines could be produced. Each target cell line would display a specific antigen epitope which allows CTL function against multiple antigens or apitopes to be determined in a single reaction. Various types of fluorescent proteins could be selected from a group consisting of green fluorescent protein (GFP), enhanced GFP (EGFP), enhanced yellow fluorescent protein (EYFP), enhanced cyan fluorescent protein (ECFP), various red fluorescent proteins from Discosoma (DsRed1, DsRed 2, DsRed Monomer, DsRed-Express), AsRed2, HcRed1 (from Heteractis crispa coral), AmCyari, ZsYellow, ZsGreen, and AcGFP-1. (BD Biosciences, Clontech, Palo Alto, Calif.). See also, U.S. Pat. Nos. 6,090,919; and 5,804,387. The various fluorescent proteins can be detected based on the different fluorescent properties, including different fluorescence wavelengths, different absorption wavelengths, and different fluorescent lifetimes.

Increased assay incubation time was beneficial to further improve the sensitivity of this FL-CTL assay. According to the fluorescence time-lapse microscopy observations, antigen specific cytolysis may not be completed until a time greater than about 2 hours after the initial contact of targets with the effectors. Increases in the FL-CTL assay incubation time (from about 4 hours, or even greater than 5 hours up to 24 hours or longer) therefore provides more opportunities for effectors to interact with targets and, correspondingly, enhances specific cytolysis. This longer incubation time is possible with the stable, lentiviral-mediated GFP and DsRed expression in viable cells that indicate similar growth rates. This similar growth rate was not observed with other FACS-based CTL assays that determine all apoptotic cells of various stages and forms (including apoptotic bodies) or use fluorescent particles as reference cells due to the different properties of target and reference populations. In addition, effector and effector-memory T cells may execute cytolytic activity more rapidly when compared to central memory CTLs which require extended periods of re-stimulation to regain their effector cytotlytic function. Therefore, by varying incubation time, this FL-CTL assay may also provide additional information, such as the proportion of effector and memory cells within the tested population, and be a valuable tool for studies of CTL effector and memory development and function.

Another important feature of this FL-CTL assay is that it is not limited to FACS-based operation. As an alternative approach, fluorescence microplate readers can also be employed. Because the fluorescent protein expressing target cells are pre-sorted to high purity with relative narrow fluorescent protein expression ranges, their absolute fluorescent intensity is directly proportional to the number of fluorescent protein-expressing cells. Using a series of controls, various numbers of the target cells are added to different wells without addition of effector cells. These wells are incubated for the same time period as the target cells with effector cells. Thus, CTL-mediated cytolysis can be determined based on the decrease in fluorescent intensity of the wells with both target and effector cells as compared to the standard curve constructed using the fluorescence from the control wells. A linear regression may be used to calculate CTL activity. Using this detection method, large numbers of samples may be analyzed simultaneously with significant improvement in efficiency compared with FACS-based determination. For this fluorescence microplate reader-based FL-CTL assay, use of a reference (R) cell line is optional. In addition, this system could use multiple target cell lines, each expressing a different fluorescent protein and displaying a specific antigen epitope. This multiple target cell system would allow CTL function against multiple antigens or epitopes to be determined in one reaction by the use of different filter systems to visualize the different proteins. This technique makes high throughput operation possible for clinical applications of large scale therapeutic evaluation and drug screening in pharmaceutical industry.

One precaution in using this FL-CTL assay is the potential interference of large numbers of input effector cells (E:T>100:1) on fluorescent intensity. To minimize the inaccuracy introduced by this factor, it is suggested to add the same number of non-antigen specific T cells to the same wells of the standard curve construction.

Of particular interest, recent advances in flow-cytometry technology and molecular engineering of increased number of diversified fluorescent proteins make it possible to further extend the capacity of this FACS-based CTL assay to simultaneous detection of CTL function against multiple antigens or epitopes. See S. C. De Rosa et al., “Beyond six colors: a new era in flow cytometry,” Nat. Med., vol. 9, pp. 112-117 (2003); and T. S. Hawley et al., “‘Rainbow’ reporters for multispectral marking and lineage analysis of hematopoietic stem cells,” Stem Cells, vol. 19, pp. 118-24 (2001). For instance, with FACSAria capable of analyzing up to 13 fluorescent colors, one set of fluorescent protein-expressing reference cells can serve as an internal control for 12 different fluorescent, color “coded” targets in the same CTL assay well, especially when the number of effectors is a limiting factor. Examples of such color proteins are listed above. This technique may also be applied to fluorescence microplate reader-based detection if filter sets are carefully designed to avoid spectral cross-interference of various fluorescent proteins.

A potential limitation of this assay is its indiscriminate measurement of direct target cell cytolysis regardless of mediating pathways. In addition, since this assay measures a relatively late event of cytolysis compared to some of the other assays examining relative early events, such as caspase activation or degranulation, its sensitivity within short incubation times may be inferior. However, increasing incubation time to greater than 2 hours brought the sensitivity to at least the same level as the other assays. Furthermore, it will be beneficial if this CTL assay system can be further extended to in vivo evaluation of CTL function.

Thus, an optimal means of standardizing FACS-based CTL assay with convenience and reproducibility, especially in industry and clinical scale-up setting, is to have two or more easily accessible and similar cell populations (only different in fluorescent spectra) for targets and references to eliminate the needs and variability of fluorophore labeling each time before use. This will be especially valuable for clinical evaluation of patients' immune responsiveness against infectious agents, such as HIV, or improvement in immune responses against tumor antigens during immunotherapy treatment. Currently, clinical evaluation of antigen specific CTL activity heavily relies on either T cell cytokine production through ELISPOT assay or conventional CTL assays that were described earlier. All those involve lengthy (days) and tedious process. Thus, a more rapid, sensitive, and convenient assay system would allow timely assessment of patients' immunological status enabling prompt initiation and re-adjustment of proper treatment regimens. Furthermore, FACS-based analysis may not be generally accessible by all the laboratories, especially in clinical settings, and more importantly, FACS-based operation often limits the number of samples to be analyzed. It is, thus, of major advancement if a CTL assay, based on the same principle, can be easily determined through other methods, such as the microplate reader-based FL-CTL, capable of simultaneous multi-sample analyses without additional and tedious preparation procedures. This microplate reader based FL-CTL would also make the screening of large numbers of cytokines or immune stimulatory agents on improvement in CTL activity easier to achieve.

### EXAMPLE 1

Materials and Methods

Animals. Four to eight week old BALB/c mice were purchased from Charles River Laboratories, Inc. (Wilmington, Del.). T cell receptor (TCR) transgenic (Tg) mice (clone 4), whose CD8 T cells specifically recognize an MHC class I epitope of influenza hemagglutinin (HA₅₁₈₋₅₂₆), were originally generated at The Scripps Research Institute (La Jolla, Calif.) and bred in the animal care facility at Louisiana State University Health Sciences Center, New Orleans, La.

Lentiviral vector construction, virus production and establishment of target (T cells) and reference (R cells) cell lines. Cppt.EF.GFP was constructed by inserting the HIV central polypurine tract to previously modified EF.GFP as described in Y. Cui et al., “Targeting transgene expression to antigen-presenting cells derived from lentivirus-transduced engrafting human hematopoietic stem/progenitor cells,” Blood, vol. 99, pp. 399-408 (2002). Using the same technique, Cppt.EF.DsRed was obtained by replacing the GFP with the DsRed2 gene (Clontech, Palo Alto, Calif.). Lentiviruses were produced by co-transfection of cppt.EF.GFP or cppt.EF.DsRed with the packaging construct pCMVdl-8.4 (kindly provided by the University of Torino, Italy) and envelope pMD.G to 293T cells using a conventional calcium phosphate precipitation method as previously described (Cui et al, 2002). P815 (mouse mastocytoma tumor cells; American Type Culture Collection, Manassas, Va.) cells were transduced with cppt.EF.GFP or cppt.EF.DsRed at a multiplicity of infection (MOI) of 5. The cells were sorted 5 days post-transduction to a purity of >98% using a FACSCalibur (BD Biosciences, San Jose, Calif.). After transduction, the cells were cultured as stable lines in the absence of additional selection.

Antibodies and flow cytometery analysis. Fluorophore-conjugated anti-mouse CD44, CD62L, CD90.1 (Thy1.1) and CD8 were purchased from PharMingen (San Diego, Calif.). FACS analysis was carried out using a FACSCalibur.

Activation of CD8⁺ HA specific cytotoxic T cells (effector “E” cells). In vitro activation of hemagglutinin (HA) specific cytotoxic T cells (CTL cells) was achieved by culturing CTL cells from clone 4 Tg mice with MHC-I HA₅₁₈₋₅₂₆ specific peptide (IYSTVASSL, 10 μg/ml) for 3 days in RPMI medium supplied with 10% fetal bovine serum (FBS), 1 mM sodium pyruvate, and 0.1 mM non-essential amino acids (Invitrogen, Carlsbad, Calif.). In vivo activated HA specific T (CTL) cells were obtained from BALB/c mice exposed to HA antigen for 3 days via immunization with recombinant vaccinia virus encoding HA gene (Vacc-HA, 1×10⁷ PFU), either in the presence or absence of adoptive transfer of 2×10⁶ HA specific T cells from Tg clone 4 mice.

Preparation of bone marrow-derived dendritic cells (BM-DC) and in vivo activation of GFP-specific CTL (effectors). Bone marrow cells harvested from BALB/c mice were transduced with lentiviral vector cppt.EF.GFP at MOI of 20 and cultured in the presence of 1000 U/ml murine granulocyte-macrophage colony-stimulating factor for 8 days following conventional procedures as described in Y.Cui et al., “Immunotherapy of established tumors using bone marrow transplantation with antigen gene-modified hematopoietic steni cells,” Nat. Med., vol. 9, pp. 952-958 (2003). The mature, GFP-expressing BM-DC (GFP-BMDC) were injected in BALB/c mice subcutaneously at 1×10⁶/mouse, followed by a booster injection of the same number of cells a week later. T lymphocytes were harvested from the spleen of these mice 3 days after the second BMDC injection and used in the following CTL assay for evaluating GFP-specific CTL function.

JAM test and ⁵¹Cr-release assay. P815 cells, either wild type or GFP transduced, were pulsed with 5 μCi/ml ³H-Thymidine and loaded with HA-I peptide (10 μg/ml) for 2-3 hours for use as target cells (“T”). After extensive washing, the cells were cultured with the above activated HA-specific T cells at various effector cell:target cell (E:T) ratio for 4 hours. All the target cells were harvested using a FilterMate cell harvester (Perkin-Elmer, Boston, Mass.), and the remaining ³H-thymidine in viable targets was determined by TopCount scintillation counter (Perkin-Elmer). HA antigen-specific cytolysis was calculated as 100%×(counts of control well—counts of experimental well)/counts of control well. ⁵¹Cr release assay was carried out as described by G. Karupiah et al., “Elevated natural killer cell responses in mice infected with recombinant vaccinia virus encoding murine IL-2,” J. lmmunol., vol. 144, p. 290 (1990). Briefly, 2×10⁶ GFP-transduced P815 cells were simultaneously loaded with HA-I peptide (10 μg/ml) and Na₂⁵¹CrO₄ at a concentration of 0.5 mCi/ml for 90 min at 37° C. These cells were washed 3 times with cold CTL medium and plated out in triplicates in a v-bottomed 96-well plate at 2×10⁴ effector cells/well with various numbers of target cells to generate different E:T ratios. After a brief centrifugation, the cells were cultured at 37° C. By the end of the 6 hour incubation, 35 μl of the supernatant was transferred from each well to a 96-well Lumaplate (Perkin-Elmer). The basal ⁵¹Cr release was determined using the supernatant of target cells alone, and the maximum releasable ⁵¹Cr was determined with the supernatant of Triton-lyzed target cells. After the Lumaplate was dried overnight, the radioactivity of released ⁵¹Cr was determined by a TopCount scintillation counter. HA antigen-specific cytolysis was calculated as 100%×(counts of experimental well—counts of basal release)/(counts of maximal releasable ⁵¹Cr-counts of basal release).

New FL-CTL assay with stable fluorescent protein expressing target and reference cells to determine HA specific CTL function. Either the established GFP or DsRed expressing P815 cell lines were loaded with MHC I HA peptide (10 μg/ml) for 2-3 hours and used as target cells (T), while other unloaded cells were used as reference (R) cells at a T:R ratio of 1:1. The T-R mix was incubated at 37° C. with various numbers of activated HA-specific T (CTL) cells (effector (E) cells) in v-bottomed 96-well plate for 4 to 24 hours.

FACS-based analysis of antigen specific cytolysis. At the end of incubation, the T-R and effector mix was harvested and washed with FACS buffer. All the viable P815 cells, which were larger in size than effectors, were gated for analysis of relative percentage of GFP⁺ vs. DsRed⁺ cells via the FACSCalibur. Antigen-specific killing was calculated as the following: (1-experimental T-R ratio/control T-R ratio)×100%.

Fluorescence, timelapse microscopy. HA loaded, GFP⁺ target, and DsRed⁺ reference cells were mixed at a 1:1 ratio in a 35 mm dish and incubated with Hoechst nuclear counterstain (Molecular Probes, Eugene, Oregon) on a temperature-controlled stage of a Leica DMRXA upright, epifluorescence microscope. Effector cells, also labeled with Hoechst dye, were added to the T (target)-R (reference) mix at E:T ratio of 20:1. Image acquisition at a rate of one frame/minute was initiated immediately upon effector addition for 3 hours through a 63× liquid immersion objective lens on the microscope, which was connected to a computer integrated Sensicam QE CCD camera. Filter sets optimized for detecting EGFP signal were exciter HQ480/20, dichroic Q495L, and emitter HQ510/20 m. Optimal filter sets used for DsRed were exicter 545/30, dichroic Q570DLP, and emitter HQ620/60 m. Filters for detecting Hoechst dye were exciter 360/40, dichroic 400DCLP, and emitter GG420LP. All filters were purchased commercially from Chroma Technology Co. (Rockingham, Vt.). Image analyses were performed with Slidebook™ software (Intelligent Imaging Innovations, Denver, Colo.).

Fluorescence microplate reader-based CTL analysis. FL-CTL assay was set-up almost identical to the FACS-based analysis, except GFP or DsRed alone was added to each well in triplicates as targets. In addition, a serial dilution of the target cells was plated out in triplicates in the same plate for a standard curve construction. At the end of a 4-hour incubation, cells were washed 3 times with phosphate buffered saline (PBS; Gibco BRL, Inc., Grand Island, N.Y.) and transferred to a 96-well flat, clear bottom, white polystyrene framed microplate (Coming). The fluorescent level of the remaining target cells was determined using a Bio-Tek (Winooski, Vt.) FL600 Fluorescence microplate reader. GFP signal was determined with a filter set of excitation wavelength at 485/20 nm and emission at 530/20 nm; and DsRed determined with a filter set of excitation at 550/20 nm and emission at 620/40 nm. The remaining number of GFP⁺ or DsRed⁺ targets in each well was calculated based on the standard curve constructed for each cell type. The antigen-specific cytolysis was determined as the following: (1-number of cells in experimental well/number of cells in control well)×100%.

### EXAMPLE 2

Lentiviral Vectors Efficiently and Stably Integrated GFP and DsRed Transgenes to P815 Cells, Without Affecting Cell Viability or Long-term Growth

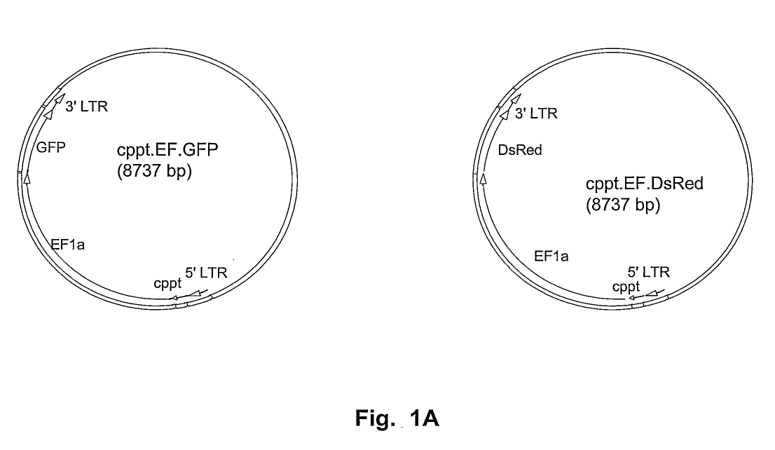

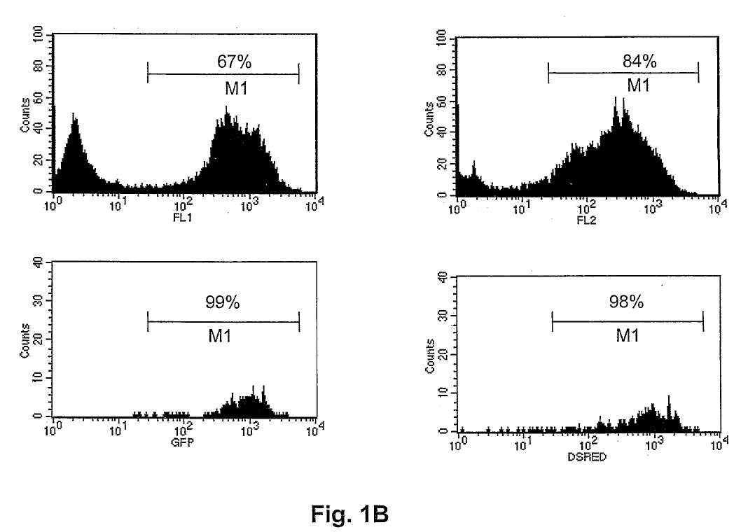

Direct measurement of target cell cytolysis requires a means of distinguishing them from other populations (e.g. conventionally used radioisotopes or recently used fluorophoric molecules). Taking advantage of efficient gene transfer and long-term expression of lentiviral vector delivery system to most of the cells, two lentiviral vectors were constructed that expressed either enhanced green fluorescence protein (EGFP) or red fluorescence protein (DsRed) driven by a promoter of the human elongation factor (EF1α), as illustrated in FIG. 1A. FIG. 1A gives a schematic illustration of cppt.EF.GFP and cppt.EF.DsRed lentiviral vectors used for transducing P815 cells. Then P815 cells were transduced with one or the other lentivirus at MOI of 5. Five days after the transduction or when the transduced cells were expanded to more than 2×10⁶, the cells were sorted to GFP⁺ or DsRed⁺ cells at a purity of >98% (FIG. 1B). Their purity and fluorescence intensity remained unchanged in long-term culture without additional selection, and were subsequently used as either target (T) or reference cells (R) (FIG. 1B).

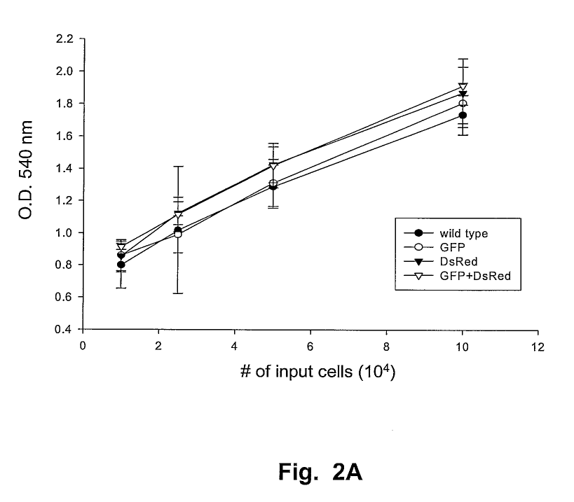

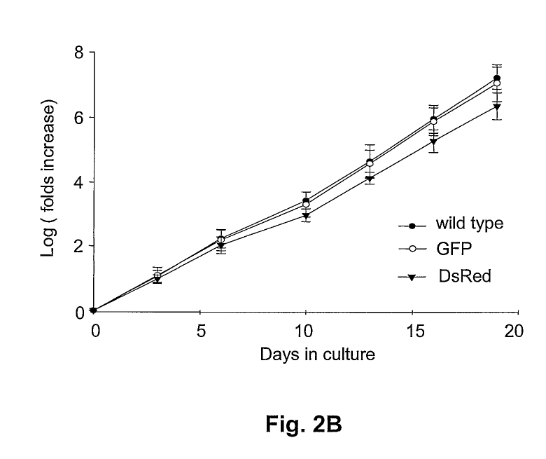

To confirm that GFP or DsRed expression in P815 cells did not affect either their viability or growth rate, a 4-hour MTT assay was conducted with either GFP⁺ cells or DsRed⁺ cells alone, or combined at a 1:1 ratio in various numbers. Metabolic activity of these cells were determined by the optical density (O.D.) reading of metabolized MTT substrate and compared with that of wild type P815 used as a control. As demonstrated in FIG. 2A, no significant differences in metabolic activity (O.D. reading) was observed among the transduced cells compared to unmodified P815 (wild type). All cells showed a linear increase in metabolic activity with input cell numbers (FIG. 2A).

Lentiviral-transduced GFP or DsRed expressing cells were cultured for 3 weeks with 1:5 subculture every 3-4 days, and their growth expressed in fold of expansion in log scale was plotted against time in culture. The data are shown in FIG. 2B, where each point is an average of 3 separate experiments with triplicate samples for each experiment. As shown in FIG. 2B, during the 3-week culture, the proliferation rate and viability of GFP⁺ or DsRed⁺ P815 cells were also not affected by the transgene expression. Thus, these two transduced cell lines behaved almost identically to each other and to the wild type in culture. This means that the cells would maintain a relatively constant ratio when mixed in both short-term and long-term culture conditions.

### EXAMPLE 3

HA-antige Specific Cytolysis of Transgene Expressing P815 Target can be Efficiently Determined by FACS Analysis and Visualized by Fluorescent Microscopy (FL-CTL Assay)

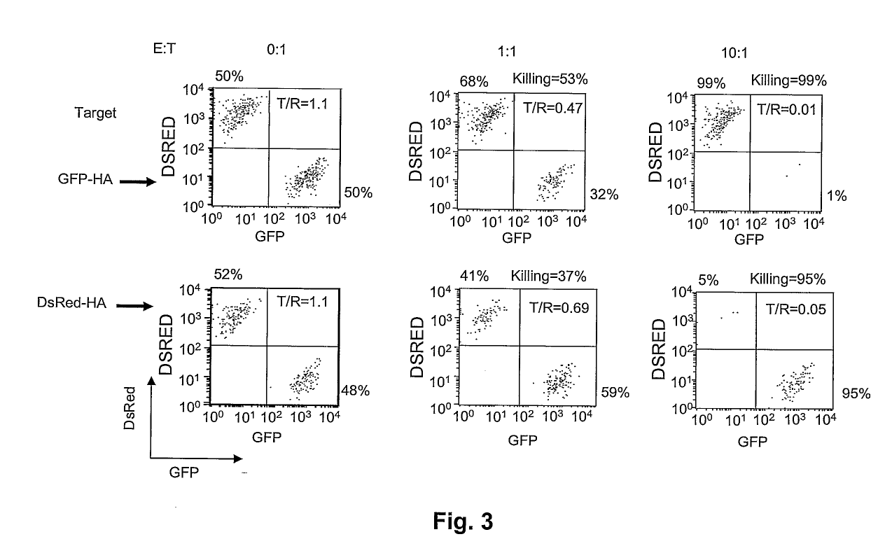

To examine whether GFP- or DsRed-expressing target cells could efficiently present antigen to cytotoxic T (CTL) cells and thus be subject to) cytolysis, either GFP⁺ or DsRed⁺ cells were loaded with an influenza hemagglutinin (HA) MHC class I peptide (10 μg/ml) as target (T). These loaded cells were then mixed with non-peptide loaded fluorescent protein (DsRed⁺ or GFP⁺, respectively) expressing cells used as reference (R) at a 1:1 ratio. This T (target)-R (reference) mix was cultured with various numbers of in vitro activated HA specific CTL effectors (E) in a V-bottomed 96-well plate for 4 hours. The relative percentage of remaining target cells (T) compared to reference cells (R) was analyzed by FACS. Antigen-specific cytolysis was calculated as (1-experimental T-R ratio/control T-R ratio)×100%. More specifically, FIG. 3 shows the stable P815 cell lines expressing GFP or DsRed that were loaded with HA specific MHC I peptide and used as targets (T) (GFP-HA, top and Red-HA, bottom, respectively) in CTL assays. The cells were mixed with reference cells P815-DsRed (top panels) or P815-GFP (bottom panels) at a 1:1 ratio and incubated with activated HA-specific CTLs (effector cells; E) at three E:T ratios of 0:1, 1:1, and 10:1. At the end of 4-hour incubation, these cells were harvested for FACS analysis, and all viable P815 cells were gated based on their size to determine changes in percentage of target cells vs. reference cells. HA specific cytolysis was calculated as (1-experimental T-R ratio/control T-R ratio)×100%. FIG. 3 represents FACS plots of five individual experiments. Interestingly, even at an E:T ratio of 1:1, specific killing of the HA peptide loaded targets reached 30-50%, regardless whether the HA presenting targets were GFP⁺ or DsRed⁺ cells (FIG. 3, middle top and bottom panels). When the E:T ratio reached 10:1, more than 95% of the target cells were eliminated by the end of 4-hour incubation. (FIG. 3, right top and bottom panels).

To directly visualize the antigen-specific cytolysis, the CTL process was monitored in a 35 mm-dish with an automated epifluorescence microscope on a temperature-controlled stage, using GFP⁺ cells as targets and DsRed⁺ cells as references. Briefly, 200,000 activated HA specific effectors were added to a mixture of HA-I loaded GFP⁺ target and DsRed⁺ reference cells at a T:R ratio of 20:1 to a 35 mm dish. Timelapse images of cell-cell interaction and cytolysis were captured during a 3-hour period as evidence of chromatin condensation and fragmentation specific to GFP⁺ cells (green cells within the white frames and enlarged images underneath). A Hoechst dye was used to label nuclei at the blue fluorescent spectrum to simultaneously visualize effectors (blue only), GFP⁺ target (green and blue), and DsRed⁺ reference (red and blue) cells (Data Not Shown). The cell-cell interaction was monitored and images captured at 1 minute/frame for 3 hours with a computer integrated CCD camera. Interestingly, brief contacts of HA-specific effectors with both GFP⁺ and DsRed⁺ cells occurred almost immediately upon the addition of effector cells. However, obvious changes, such as chromatin condensation and bleb formation (i.e. signs of apoptosis), were only observed in HA-presenting GFP⁺ cells starting at around 1 hour after the initial interaction. (Data not shown) By three hours after the E-T interaction, GFP⁺ targets exhibited evident DNA fragmentation and cell morphology changes, while DsRed⁺ cells remained unchanged. (Data not shown) However, the CTL-mediated target cell cytolysis observed under the fluorescent microscope required a longer time when compared to the FL-CTL and other conventional CTL assays due to the reduced chance and random interactions of effector and target cells in the 35-mm dish used for the microscope as compared to the v-bottomed 96-well plate used in the other assays.

### EXAMPLE 4

This FL-CTL Assay is Specific and Sensitive in Evaluating HA Specific CTL function

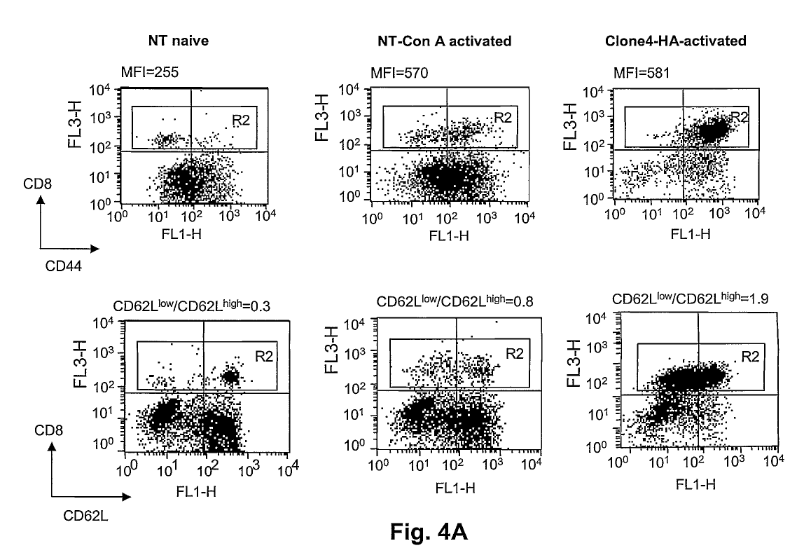

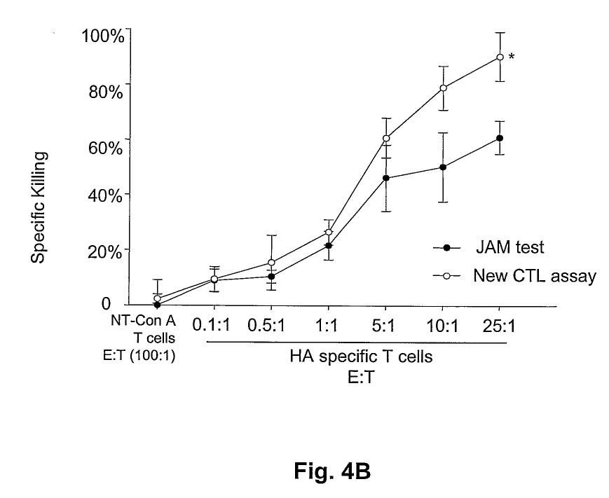

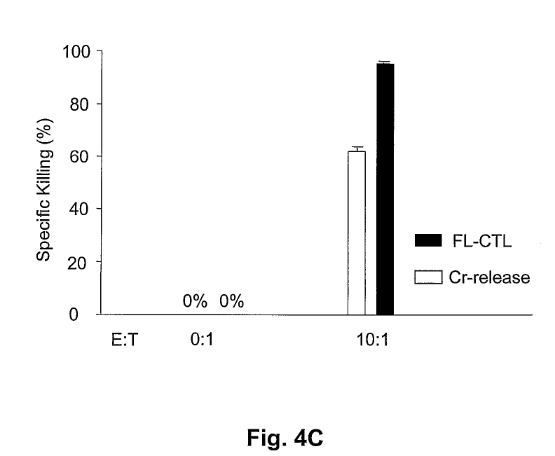

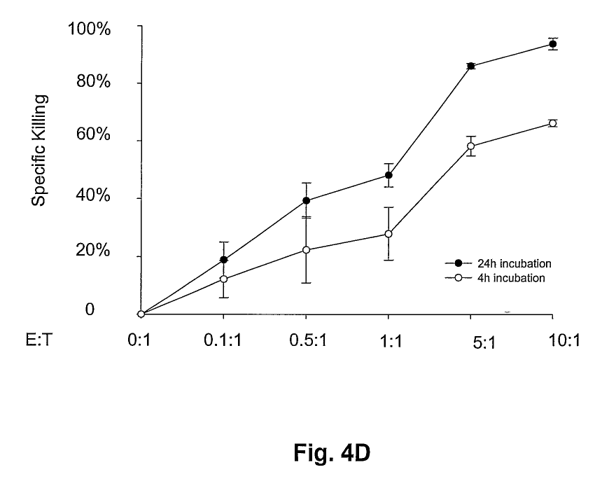

To answer the question of whether the specificity and sensitivity of this FL-CTL assay were comparable to those of conventional assays, two types of activated CTL cells were prepared, Con-A stimulated HA non-specific CTL cells from non-transgenic (NT) mice and HA peptide-stimulated CTL cells from TCR transgenic mice (clone4), whose activation was confirmed by up-regulation of CD44 and down-regulation of CD62L. CTL cells from non-transgenic mice (NT) were activated via 3-day Con A stimulation, and HA specific CTL effectors were activated by MHC-I HA peptide in 3-day culture. The CTL cell activation status was characterized as up-regulation of CD44 or down-regulation of CD62L expression compared to non-activated cells. In FIG. 4A are FACS plots of these experiments indicating the activation status of these CTL cells. Since cytotoxic T cells acquire CTL function only upon activation, this example confirmed that the lack of HA specific CTL function of ConA activated CTL cells as described in the following experiment was not the result of insufficient activation of the CTL cells.

These activated CTL cells were each used as effectors in the FL-CTL assay (using GFP⁺ cells as targets) and also in the conventional JAM test (³H-thymidine pulsed GFP⁺ cells as targets), at various E:T ratios. The results are shown in FIG. 4B, where each point is a mean±S.E. (N=3). An asterisk indicates a significant difference of P<0.05 as calculated by ANOVA. An E:T ratio of 100:1 resulted in minimal cytolysis in both assay systems. Interestingly, when activated HA-specific CTL cells were used as effectors, cytolysis of GFP⁺ targets was reproducibly detected by FL-CTL assay even at an E:T ratio of 0.5:1 (FIG. 4B). An incremental increase in killing with the increase in E:T ratio was observed, which reached up to 100% at E:T ratios between 10:1 and 25:1 (FIG. 4B). In contrast, HA specific cytolysis as determined by the JAM test reached only 50-60% at an E:T ratio of 25:1 (FIG. 4B).

The FL-CTL assay was also compared with the conventional ⁵¹Cr release assay in determining CTL activity of in vitro activated HA specific T cells at the E:T ratios of 0:1 and 10:1. FIG. 4C shows the results, and each point represents mean±S.E. (N=3). In FIG. 4C, some error bars may not be easily identifiable due to the small number. Both assays showed almost no non-specific lysis of target cells in the absence of activated HA specific CTL cells. (E:T of 0:1) However, when 2×10⁵ HA specific CTL cells were cultured with 2×10⁴ targets for 6 hours (an E:T of 10:1), about 60% of the targets were eliminated in the ⁵¹Cr release assay, whereas around 90% of specific killing was detected by the new FL-CTL assay (FIG. 4C).

In addition, as neither GFP nor DsRed expression altered cell viability and growth rate during an extended culture period, an increase in the duration of this FL-CTL assay could reasonably enhance effector interaction with target cells and subsequently increase the assay sensitivity. As expected, the level of specific cytolysis determined in a 24-hour vs. standard 4-hour incubation and thus the sensitivity, was significantly enhanced without an obvious increase in non-specific killing (FIG. 4D). Again, FIG. 4D represents the mean±S.E. (N=3), where an * represents a significant difference between the two ratios at P<0.05 as analyzed by an ANOVA.

The CTL cells used in FIGS. 4C and 4D were generated under slightly different conditions, and thus CTL activity, expresses as percentage, may be different. As shown in FIG. 4C, the new FL-CTL assay is more sensitive than the conventional ⁵¹Cr assay. FIG. 4D indicates that FL-CTL sensitivity can be enhanced by increasing the assay incubation time.

### EXAMPLE 5

Antigen Specific Cytolysis with the FL-CTL Assay Determined Via a Fluorescence Microplate Reader

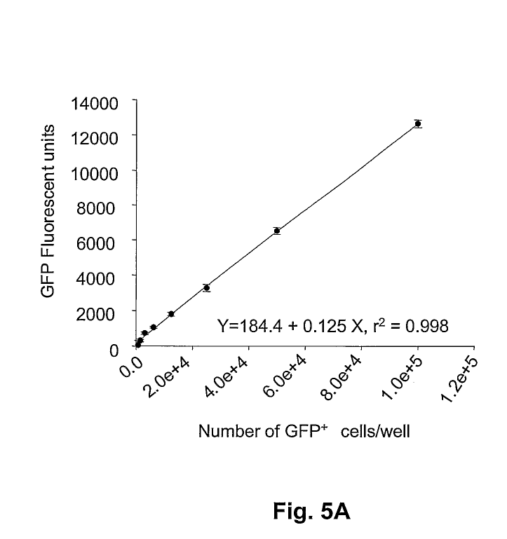

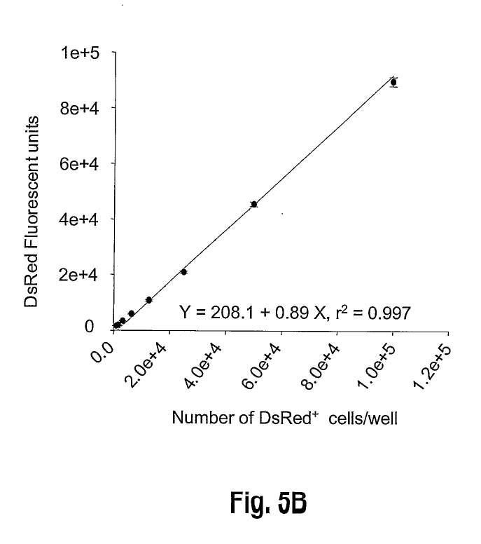

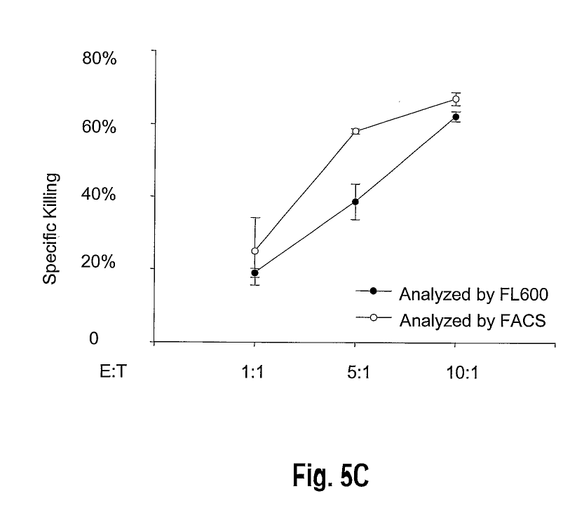

An experiment was conducted to examine whether a close relationship between the numbers of GFP⁺ or DsRed⁺ cells and their total fluorescent intensity could be established using a Bio-Tek FL600 fluorescence microplate reader, since the GFP⁺ and DsRed⁺ cells were sorted populations with relatively narrow ranges of fluorescent intensity. GFP⁺ or DsRed⁺ P815 cells were serially diluted in 96-well plates, and their fluorescent level was determined using a FL600 microplate fluorescence reader. The results are shown in FIGS. 5A and 5B. Statistical analysis determined the linear relationship between input cell number and fluorescent intensity. Indeed, a robust linear correlation (r²>0.997) of fluorescent intensity vs. numbers of input GFP or DsRed⁺ cells was shown to exist, as determined with filter set wavelength of excitation at 485/20 nm and emission at 530/20 nm for GFP and a set of excitation at 550/20 nm and emission at 620/40 nm for DsRed, respectively. (FIGS. 5A and 5B, respectively).

The sensitivity and reliably of this fluorescence microplate reader-based FL-CTL detection procedure was then compared to that of the FACS-based detection. To minimize the impact of cell division during the assay-incubation period on absolute fluorescent level of either GFP⁺ or DsRed⁺ cells, a serial dilution of the same target cells (DsRed⁺ P815 cells) was seeded in the same plate at the time of CTL assay set-up, and the input cell number was used to construct a standard curve of cell number vs. fluorescent level. The number of remaining GFP⁺ or DsRed⁺ target cells post-cytolysis was calculated against this standard curve. Cytotoxicity was determined as follows: (1-number of cells in experimental well/number of cells in control well)×100%. Each point in FIG. 5C represents a mean±S.E. (N=3). Clearly, this microplate-reader-based CTL assay reliably detected 20% cytolysis at an effector (E): target (T) ratio of 1:1, which was very similar to the results obtained with the FACS-based determination (FIG. 5C). More importantly, a proportional increase in cytolysis activity with the increased E:T ratio again confirmed the reliability and specificity of this fluorescence microplate reader-based FL-CTL detection method.

### EXAMPLE 6

This FL-CTL Assay is Sensitive and Convenient for Evaluation of in Vivo Activated Antigen Specific CTL in Different Antigen Systems Without Further in Vitro Stimulation

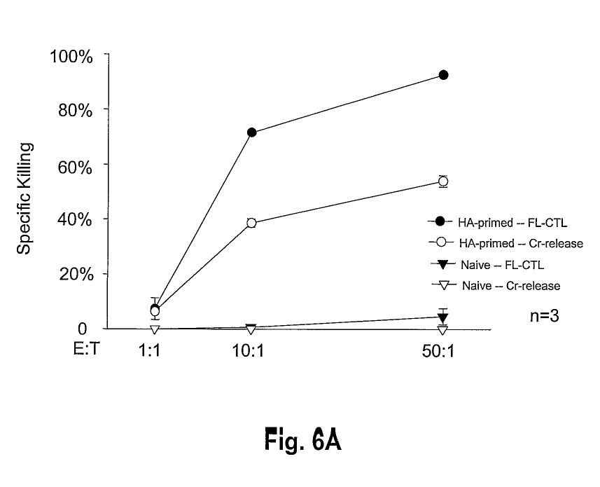

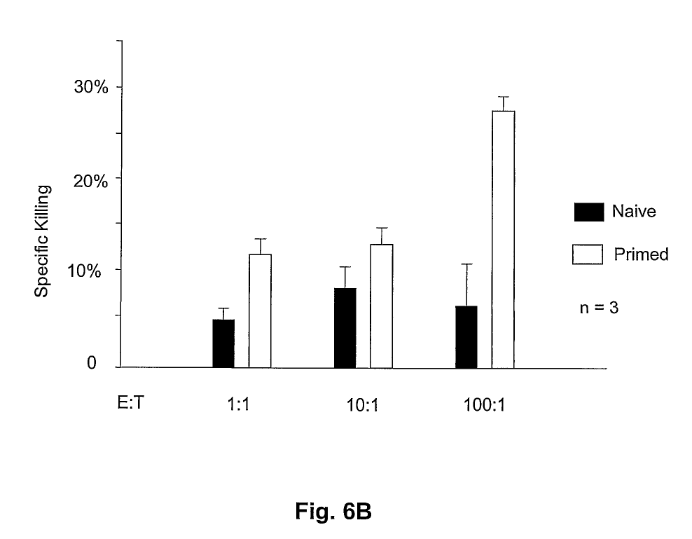

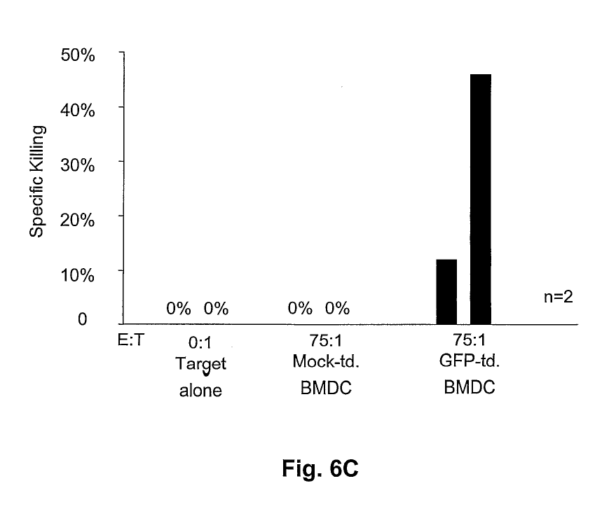

Since in vivo activated T cells are usually found at a much lower frequency than in vitro activated T cells, their further in vitro expansion/stimulation is usually required before CTL function can be evaluated with conventional assays. To test whether the increased sensitivity of the FL-CTL assay allowed a direct evaluation of CTL function of in vivo activated T cells, first naïve HA specific T cells were adoptively transferred from clone 4 Tg mice to non-transgenic mice and then immunized with 1×10⁷ vaccinia virus expressing HA antigen (Vacc-HA, HA-primed). Three days after activation with Vacc-HA, in vivo activated HA specific T cells (splenocytes) were harvested from the mice. These CTL cells were then used in a side-by-side study as effectors (E) for both FL-CTL and ⁵¹Cr release assays with HA-loaded GFP⁺ cells as targets (T) at E:T ratios of 1:1 10:1 and 50:1, using the same number of T cells harvested from unprimed naïve mice as negative controls (FIG. 6A) Each point in FIG. 6A represents a mean±S.E. (N=3). Interestingly, at an E:T ratio of 1:1, both assays showed 5-10% of HA specific CTL cytolysis (FIG. 6A). When the E:T ratios were increased to 10:1 and 50:1, dose related increases in HA specific cytolysis were observed with both assays (FIG. 6A). However, in all the E:T ratios examined, the FL-CTL assay always indicated higher CTL lysis than when compared with that determined by ⁵¹Cr release assay (FIG. 6A). For example, at an E:T ratio of 50:1, the specific killing % for the FL-CTL assay was about 90%, while the ⁵¹Cr release assay only indicated about 50%.

To further verify that this FL-CTL assay is sensitive enough in determining CTL activity from endogenous CTL cell repertoire activated by immune stimulation, T cells were harvested from Vacc-HA immunized mice in the absence of adoptive transfer of HA specific CTL cells, and used as effector cells (E). Again, HA-loaded GFP⁺ cells were used as targets (T), and the FL-CTL assay was done using E:T ratios from 1:1 to 100:1. The results are shown in FIG. 6B, where each bar represents the mean±S.E. (N=3). As shown in FIG. 6B, even with HA specific effector cells activated from an endogenous source, the FL-CTL assay reliably detected HA specific cytolysis at an E:T ratio of 100:1 (FIG. 6B).

Finally, to verify that this FL-CTL assay system can be generally applied to examine CTL function against other defined antigens, such as GFP, BALB/c mice were primed and boosted with 1×10⁶ lentiviral vector GFP transduced bone marrow derived dendritic cells (GFP-BMDC), each time at 7 days apart. As a control, another group of mice were injected twice with the same number of mock-transduced BMDC. Three days after the second injection, CTL cells (splenocytes) were harvested from these immunized mice. GFP specific CTL cells were examined using GFP⁺ P815 cells as target and DsRed⁺ P815 cells as reference with two mice in each group. As expected, specific cytolysis of GFP+ targets was only detected with CTL cells harvested from GFP-BMDC immunized mice, but not from mice immunized with mock-transduced BMDC (FIG. 6C). Thus, the FL-CTL assay was able to evaluate antigen specific CTL function of both in vitro and in vivo activated T cells in various antigen systems.

The complete disclosure of all references cited in this specification are hereby incorporated by reference. Also incorporated by reference are the complete disclosure of the following: Kong Chen et al., “FL-CTL assay: fluorolysometric determination of cell-medicated cytotoxicity using Green Fluorescent Protein and Red Fluorescent Protein expressing target cells,” accepted by the Journal of Immunological Methods, 2005 (In press). In the event of an otherwise irreconcilable conflict, however, the present specification shall control.

What is claimed:

1. A method for measuring the activity of a population of cytotoxic T lymphocytes against cells that display a selected epitope on the cell surface; said method comprising:
(a) incubating the cytotoxic T lymphocytes with a mixture of target cells and reference cells; wherein the physiology of the target cells and the physiology of the reference cells are approximately identical, except that the target cells and the reference cells express different fluorescent proteins having different fluorescence properties, and except that the target cells display the selected epitope on the cell surface while the reference cells do not display the selected epitope on the cell surface; and
(b) observing the ratio of fluorescence properties of the different fluorescent proteins at different times;
whereby:
any change in the ratio of fluorescence properties at different times is a measure of the activity of the cytotoxic T lymphocytes against cells that display the selected epitope on the cell surface.

2. A method as in claim 1, wherein at least one of the fluorescent proteins is selected from the group consisting of GFP, EGFP, EYFP, ECFP, DsRed1, DsRed 2, DsRed Monomer, DsRed-Express, AsRed2, HcRed1, AmCyan, ZsYellow, ZsGreen, and AcGFP-1.

3. A method as in claim 1, wherein the fluorescence properties are measured by flow cytometry.

4. A method as in claim 1, wherein said incubating step is at least about two hours.

5. A method as in claim 1, wherein said incubating step is greater than about five hours.

6. A method as in claim 1, wherein the initial mixture comprises approximately equal numbers of reference cells and target cells.

7. A method as in claim 1, wherein the mixture of target cells and reference cells comprises a plurality of different types of target cells, wherein each type of target cell expresses a different fluorescent protein having different fluorescence properties, and wherein each type of target cell displays a different selected epitope on the cell surface; whereby the activity of the cytotoxic T lymphocytes against the different selected epitopes is measured.

8. A method for measuring the activity of a population of cytotoxic T lymphocytes against cells that display a selected epitope on the cell surface; said method comprising:
(a) incubating the cytotoxic T lymphocytes with target cells, wherein the target cells express a fluorescent proteins, and wherein the target cells display the selected epitope on the cell surface; and
(b) observing the fluorescence properties of the fluorescent protein at different times, wherein the fluorescence properties are compared to those of a control population of target cells that is otherwise treated similarly, but that is not incubated with cytotoxic T lymphocytes;
whereby:
any change in the ratio of fluorescence properties of the target cells and control cells at different times is a measure of the activity of the cytotoxic T lymphocytes against cells that display the selected epitope on the cell surface.

9. A method as in claim 8, wherein the fluorescence properties are measured with a fluorescence microplate reader.

10. A method as in claim 8, wherein at least one of the fluorescent proteins is selected from the group consisting of GFP, EGFP, EYFP, ECFP, DsRed1, DsRed 2, DsRed Monomer, DsRed-Express, AsRed2, HcRed1, AmCyan, ZsYellow, ZsGreen, and AcGFP-1.

11. A method as in claim 8, wherein said incubating step is at least about two hours.

12. A method as in claim 8, wherein said incubating step is greater than about five hours.

13. A method as in claim 8, wherein the cytotoxic T lymphocytes are incubated with a plurality of different types of target cells, wherein each type of target cell expresses a different fluorescent protein having different fluorescence properties, and wherein each type of target cell displays a different selected epitope on the cell surface; whereby the activity of the cytotoxic T lymphocytes against the different selected epitopes is measured.
